(19) United States
(12) Patent Application Publication
(10) Pub. No.: US 2003/0008015 A1
(43) Pub. Date: Jan. 9, 2003

(54) Polymer controlled delivery of a therapeutic agent

(76) Inventors: Catherine S. Levisage, Baltimore, MD (US); Bernard A. Malavaud, Toulouse (FR); Michael F. Haller, Baltimore, MD (US); Kam Leong, Ellicott City, MD (US)

Correspondence Address: Dike, Bronstein, Roberts & Cushman, Intellectual Property Practice Group, EDWARDS & ANGELL, LLP, P.O. Box 9169, Boston, MA, 02209, US

(21) Appl. No.: 09/975,565

(22) Filed: Oct. 11, 2001

Related U.S. Application Data
(60) Provisional application No. 60/239,498, filed on Oct. 11, 2000.
(60) Provisional application No. 60/239,385, filed on Oct. 11, 2000.

(51) Int. Cl. A61K 9/50

(52) U.S. Cl. 424/501

(57) ABSTRACT

Featured are highly useful pharmaceutical compositions that include a microparticle having a polymeric support material preferably adapted to disperse therapeutic agent. In a particular invention embodiment, the support material comprises at least 50% w/w of a homopolymer as described herein. Also featured are methods of making the compositions as well as using same to treat diseases such as bladder cancer.

This application claims the benefit of U.S. Provisional Applications Serial Nos. 60/239,498 and 60/239,385 both filed Oct. 11, 2000, the teachings of which are incorporated herein by reference.

### BACKGROUND OF THE INVENTION

1. Field of the Invention

The present invention generally relates to polymers, delivery vehicles, and methods of use thereof. In one aspect, the invention features polymeric microparticle delivery vehicles for controlled administration of a therapeutic or prophylactic compound. The invention has a wide spectrum of important applications including providing for localized administration of a pharmaceutical composition such as an anti-cancer agent.

2. Background

There is recognition that many therapeutic agents can be administered with a delivery vehicle or vector to facilitate uptake. In many cases, poor solubility of the therapeutic agent in an aqueous medium such as water, blood, or saline, and the like can limit drug delivery and efficacy. For example, compounds with low water solubility are frequently formulated with a solubilizing agent. Many prior delivery vehicles have undesirable side effects including exhibiting high toxicity or acting as a sensitizing agent.

Paclitaxel is a diterpenoid natural product that is reported to belong to the class of antimicrotubule agents. There are further reports that it prevents tubule depolymerization, an important step of the cell mitosis. It has been disclosed as being a potent anticancer and antiangiogenic agent e.g., in the treatment of ovarian and breast cancer and of AIDS-related Kaposi's sarcoma. Despite its high efficacy in cell culture of bladder cancer cell lines, paclitaxel is typically not used for intravesical administration.

There is understanding in the field that paclitaxel is a highly lipophilic drug. It is believed to have poor water solubility; a feature that inhibits use as a therapeutic agent for intravesical chemotherapy. An FDA-approved formulation generally requires the use of a vehicle that causes acute toxicity after intravenous administration.

The vast majority of transitional cell carcinomas (TCC) are diagnosed at a superficial stage. Typically, the diagnosis is made when the lamina propria is intact or has just been passed through. After resection, different protocols of intravesical therapy have been proposed to reduce the number of recurrences and the risk of subsequent procession. To achieve optimal results, the drug should come in contact with the target urothelium and repeated administrations are required to obtain a response. However, there are reports that about 50% of the patients treated by complete resection and intravesical instillations of Mitomycim C or Bacille Calmotte-Guerin (BCG) had tumor resections. Of those individuals, 10% exhibited tumor progression. Subsequently half of those in progression were reported to die from cancer resulting in a 5.8% specific fatality rate. This result emphasizes the need for more effective intravesical treatment.

It thus would be desirable to have new polymers, delivery vehicles and methods of use thereof that can successfully deliver a variety of useful agents to a pre-determined biological location. It would be especially desirable to have polymeric microparticle delivery vehicles that can associate with a desired therapeutic agent and release same in a controlled manner to the location.

### SUMMARY OF THE INVENTION

The present invention generally relates to polymers, delivery vehicles, and methods of use thereof. In one aspect, the invention features polymeric microparticle delivery vehicles for controlled administration of a therapeutic or prophylactic compound. The invention has a wide spectrum of important applications including providing for localized administration of a pharmaceutical composition such as an anti-cancer agent to or near a tumor.

The invention more specifically relates to pharmaceutical compositions comprising a delivery vehicle and a therapeutic agent encapsulated within the delivery vehicle. In one embodiment, the delivery vehicle is a microparticle composed essentially of a polymer support material capable of encapsulating a therapeutic or prophylactic agent. The delivery vehicle preferably includes a polymer support material that is able to release the encapsulated therapeutic agent in a controlled process, preferably without affecting the biological activity of the therapeutic agent. Preferred practice of the invention provides a continuous (or near continuous) release of the therapeutic agent from the pharmaceutical composition. In preferred invention embodiments, the delivery vehicle of the present invention includes a polymer support material that is generally biocompatible, non-toxic and non-sensitizing.

Significantly, the invention provides for successful administration of a therapeutic agent such as a lipophilic compound or a composition that has low aqueous solubility. That agent can be administered as part of a pharmaceutical composition. Typically, the agent is encapsulated within microparticles for local delivery to a pre-determined target tissue. It has been found that many therapeutic agents retain substantial biological activity after the encapsulation process and subsequent release from the microparticle delivery vessel. It has also been found that the invention can provide for delivery of a relatively high concentration of therapeutic agent to the target (continuously or near continuously) without systemic distribution of the therapeutic agent.

In particular, it has been discovered that the important anti-cancer drug paclitaxel can be successfully loaded into microparticles of this invention and then administered intravesically to mice with BBN-induced bladder cancer. The survival rate and body weight of mice were significantly higher for mice receiving such microparticle delivery vessels with encapsulated paclitaxel than for (control) mice treated with non-loaded microparticle delivery vessels or treated with free paclitaxel.

Significantly, pharmaceutical compositions comprising paclitaxel encapsulated in microparticles composed primarily of poly(methylidene malonate 2.1.2) were unexpectedly effective at treating bladder cancer in mice such that mice treated with a single administration of encapsulated paclitaxel had a lower mortality rate and higher body weight than mice treated with multiple administrations of non-encapsulated paclitaxel.

Accordingly, and in one aspect, the invention features pharmaceutical compositions that include at least one encapsulated therapeutic agent dispersed within a microparticle composed primarily of at least one polymer support material. For example, in one embodiment, the microparticle includes a polymeric support material preferably adapted to disperse a desired substance, in which the support material comprises at least about 50% by weight of at least one homopolymer.

In a more particular embodiment of the pharmaceutical composition, the homopolymer has a repeat unit according to Formula (1):

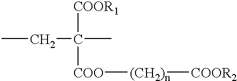

wherein

R₁ represents a C₁-C₆ alkyl group or a group (CH₂)ₘ—COOR₃ wherein m is an integer from 1 to 5 and R₃ is a C₁-C₆ alkyl group the same or different from R₁;

R₂ represents a C₁-C₆ alkyl group the same or different from R₁ and R₃;

n is an integer from 1 to 5; and

a therapeutic agent that is encapsulated or dispersed in the support material of the microparticle delivery vehicle.

Also featured are methods for the targeted, i.e. localized or semi-localized, treatment of a disease or disorder. In one embodiment, the methods include the step of administering a pharmaceutical composition with at least one encapsulated therapeutic agent dispersed in microparticles. In this example of the invention, the agent is dispersed to or near the site of the disease or disorder. In preferred practice, the microparticles localize and adhere to the cellular surfaces where the encapsulated therapeutic agent is delivered by a controlled release from the microparticle. A single application of a pharmaceutical composition of the present invention is at least as effective and in some instances much more effective than multiple applications of a non-encapsulated therapeutic agent.

Pharmaceutical compositions of the present invention can be prepared in accord with one or a combination of strategies.

For example, and in a particular preparation method according to the invention, the compositions are prepared in a single emulsification procedure. In one example, the therapeutic agent is typically dispersed within the polymer support material of a microparticle by a method that includes at least one and preferably all of the following steps:

a) preparing a first solution in a volatile organic solvent in which the solution comprises a polymeric support material and a therapeutic agent;

b) preparing a second solution immiscible with the first solution, the second solution further including a stabilizing agent;

c) preparing an emulsion by combining the first and second solutions sufficient to form a single phase of a polymer solution; and

d) evaporating the volatile organic solvent from the polymer solution to form the pharmaceutical composition.

The invention also includes use of compositions of the invewntion to treat against various disorders and diseases, particularly for treatment of or preparation of a medicament for treatment or prevention of a urological disease or disorder, including a cancer, particularly bladder cancer. Subjects that may be treated in accordance with therapies disclosed herein include mammals, particularly primates such as humans that may be suffering from or susceptible to (prophylactic treatment) such diseases or disorders.

Other aspects and embodiments of the invention are discussed below.

### BRIEF DESCRIPTION OF THE DRAWINGS

For a fuller understanding of the nature and desired objects of the present invention, reference is made to the following detailed description taken in conjunction with the accompanying drawing figures wherein like reference character denote corresponding parts throughout the several views and wherein:

FIG. 1 is a bar graph of MBT-2 cell growth in the presence of paclitaxel in culture medium. The concentration of free paclitaxel ranged from 2.9×10⁻⁶ M (left) to 2.9×1O⁻⁹ M (right). Dilution 1 through dilution 5 indicate 10-fold serial dilutions of particles suspension in culture medium;

FIG. 2 is a bar graph of MBT-2 cell growth in the presence of free paclitaxel (2.28×10⁻⁸ M (left) to 2.28×10⁻⁶ M (right)) or loaded microparticles (2.28×10⁻⁷ and 2.28×10⁻⁶ M)

FIG. 3 is a series of photographs of MBT-2 cells incubated with free paclitaxel or microparticles that are loaded with paclitaxel or are non-loaded;

FIG. 4 is a series of photographs depicting the localization of fluorescent particles on mice bladder sections (A: 3 hours and B: 48 hours) or non-fluorescent particles with a scanning electron microscopy (C: 30 minutes);

FIG. 5 is a plot of the survival (%) of mice receiving free paclitaxel, encapsulated paclitaxel or unloaded microparticles;

FIG. 6 is a bar graph of the last body weights of mice receiving free paclitaxel, encapsulated paclitaxel or unloaded microparticles;

FIG. 7 is a bar graph of the size distribution of microspheres comprising paclitaxel;

FIG. 8 is a Scanning Electron Microscopy image of PMM 2.1.2 microparticles encapsulating paclitaxel;

FIG. 9 is a plot of the cumulative release of Paclitaxel from PMM 2.1.2 microparticles in PBS containing 0.05° A of Tween 80 as a function of time (bottom); and

FIG. 10 is a plot of inhibition of MBT-2 cells growth in presence of free, extracted or encapsulated paclitaxel where cell growth was measured after 3 days of incubation and results expressed as a percentage on inhibition complied to the growth of cells incubated with medium.

### DETAILED DECRIPTION OF THE INVENTION

As discussed, the invention provides highly useful pharmaceutical compositions in which a therapeutic agent is dispersed in a microparticle delivery vehicle. Also provided are methods of using same as well as methods for preparing the pharmaceutical compositions.

In particular embodiments according to the invention, the microparticles essentially include a poly(methylidene malonate 2.1.2) polymer support material although other polymer support materials as described below may be more suitable for other applications. Typically, that material is capable of encapsulating one or more substances. More particular microparticles release encapsulated substances into the surrounding environment with a controlled rate of release. Additionally preferred microparticles do not significantly inhibit biological activity of the encapsulated substance. Additionally the present invention provides microparticles with one or more encapsulated therapeutic agents for a controlled and localized delivery of the encapsulated agents to a targeted tissue of a patient.

As used herein, the term “microparticle” is intended to include nearly any particle with a mean diameter or particle size in the range of 0.5 μm to 100 μm, with a preference for particles with a mean diameter or particle size in the range of 1 μm to 20 μm which is composed of an approximately homogenous network of the support material. Preferred microparticle geometries are spherical, ellipsoidal and the like. Other polymeric devices included within the term microparticle include but are not limited to nanoparticles, micro or nanocapsules, hydrogels, gels and the like which are capable of encapsulating, or adsorbing or complexing compounds.

It is further intended that the term microparticle refer to particles prepared by an emulsion process that can encapsulate one or more discrete globules or droplets of a substance or a mixture of two or more substances during microparticle formation. The mass fraction of the encapsulated substance(s) is preferably about 0.5% to about 20% w/w of the microparticle.

As mentioned, the present invention includes pharmaceutical compositions that include a therapeutic agent encapsulated or otherwise dispersed in a polymer microparticle delivery vehicle, a more efficient application of the therapeutic agent is achieved, especially to the site of desired treatment when compared to a suitable control, e.g. application of non-encapsulated therapeutic agent (e.g. without a delivery vehicle).

In a preferred embodiment, the pharmaceutical composition comprises:

a microparticle that includes a polymeric support material in which a substance can be dispersed, wherein the support material comprises at least about 50% w/w of at least one homopolymer with a repeat unit according to Formula (I):

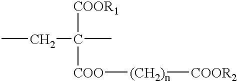

wherein

R₁ is ethyl;

R₂ is ethyl;

n is 1; and

at least one therapeutic agent, preferably paclitaxel, that is encapsulated or dispersed in the polymeric support material of the microparticle.

As discussed, a preferred polymer support material is poly(methylidene malonate 2.1.2).

By the term “alkyl” is meant a straight chain or branched chain hydrocarbon such as methyl, ethyl, propyl, iso-propyl, butyl, iso-butyl, tert-butyl, pentyl, hexyl, and the like.

In other particular examples of the foregoing microparticle, the polymer support can include polymers in which R₁ and R₂ are each independently C₁-C₆ alkyl groups the same or different; and n=1.

Additionally preferable are polymer support materials as above-described with a molecular weight (M_{W}) between 5,000 and 500,000 as determined e.g., by gel permeation chromatography, membrane osmosis, light scattering, sedimentation centrifugation or electrophoresis. More preferable polymers have a molecular weight (M_{W}) between 10,000 and 100,000. Particularly preferable polymers have a molecular weight (M_{W}) between 20,000 and 40,000. Use of a specific polymer support material will be guided by recognized parameters such as intended use.

In certain embodiments of the present invention, the polymer support material is a polymer mixture comprising at least one and typically both of the following:

from about 90 to about 99.5% by weight of at least one homopolymer as defined generally in Formula (I); and

from about 0.5 to about 10% by weight of at least one polymer additive.

Accordingly, microparticles that include more than one homopolymer, more than one additive, and/or more than one therapeutic agent are within the scope of this invention.

Preferred polymer additives comprise at least one hydrophilic compound, preferably at least one of polyethyleneoxide, polyvinylalcohol, polyvinylpyrrolidone, poly(N-2-hydroxypropyl methacrylamide), polyhydroxyethylmethacrylate, hydrophilic poly(aminoacid) such as polylysine or a polysaccharide. A particularly preferred polymer additive is polyvinylalcohol (PVA) with 0.5 to 10% w/w PVA blended into the homopolymer or more preferably 1 to 5% w/w PVA. A polymer blend with 2% PVA and 98% homopolymer is particularly preferred.

In other preferred embodiments of the present invention, pharmaceutical compositions can include substances that are encapsulated or otherwise dispersed in a polymeric delivery vehicle that are hydrophobic, hydrophilic or require a solvation vehicle for administration wherein the requirement can be derived from poor solubility, undesirable therapeutic degradation pathways and the like that inhibit the effective delivery of a therapeutic without a delivery vehicle.

In specific embodiments the therapeutic agent can be a drug, a therapeutic agent, an anticancer agent, a gene therapy agent, a plasmid, DNA, a protein, an enzyme, a peptide, a radionuclide, a protein inhibitor, an analgesic, an anti-inflammatory agent, an antibiotic, an antiviral agent, an antineoplastic agent, 5-FU, a cytotoxic agent, an immunomodulator, a hormone, an antibody or a painkiller. Additionally, a mixture of two or more therapeutic agents can be encapsulated in the microspheres for applications where synergistic drug effects are desirable.

In particularly preferred embodiments the dispersed therapeutic agent is an anticancer agent or a gene therapy agent. Paclitaxel, docetaxel (taxotere®), taxol® and other members of the taxane family of anticancer agents are preferred chemotherapy therapeutic agents for the treatment of urological diseases or disorders, specifically bladder cancers. Other suitable anticancer agents include recognized chemotherapeutic or anti-neoplastic agents, particularly alkylating agents, anti-metabolites, natural agents, hormones and hormone antagonists and miscellaneous products as described by Calabusi, P. and R. E. Parks Jr. (1985) in the Pharmaceutical Basis of Therapeutics, Chpt. XIII MacMillan Publishing Co. (New York), The disclosure of which is incorporated herein by reference.

Preferred antimetabolites for use in accord with this invention include analogs of folic acid (e.g., methotrexate), pyrimidine analogs (e.g., fluorouracil, cytarabine) and analogs of purine (e.g., mercaptopurine and thioguanine). Acceptable gene therapy agents according to the invention include a specific nucleic acid sequence that encodes a protein or polypeptide having desired therapeutic or cytotoxic activity.

Pharmaceutical compositions comprising a hydrophobic or lipophilic therapeutic agent of the present invention can be prepared by one or a combination of different strategies as described herein including at least one and preferably all of the following steps:

a) preparing a first solution in a volatile organic solvent wherein the solution comprises a polymeric support material and a therapeutic agent;

b) preparing a second solution immiscible with the first solution, the second solution further including a stabilizing agent;

c) preparing an emulsion by combining the first and second solutions sufficient to make a single phase being that includes a polymer solution; and

d) evaporating the volatile organic solvent, preferably while stirring the emulsion, to make the pharmaceutical composition.

Pharmaceutical compositions comprising a hydrophilic therapeutic agent of the present invention can also be prepared by one or a combination of different methods including at least one and preferably all of the steps in the following method, or it can also be prepared by a double emulsion method:

a) preparing a first solution in a volatile organic solvent wherein the solution comprises a polymeric support material;

b) preparing a second aqueous solution immiscible with the first solution, the second solution further including a stabilizing agent and the therapeutic agent;

c) preparing an emulsion by combining the first and second solutions sufficient to make a single phase that includes a polymer solution; and

d) evaporating the volatile organic solvent, preferably while stirring the emulsion, to make the pharmaceutical composition.

For the double emulsion method, the hydrophilic therapeutic agent is dissolved is water, emulsified in an organic solvent with or without an emulsifier, and then the resulting emuslion is further dispersed in an aqueous solution with an emulsifier, to create a water-in-oil-in-water mixture. The microspheres will then be prepared in a similar manner as described above.

Additionally preferred pharmaceutical compositions include a therapeutic agent encapsulated or otherwise dispersed in the polymer support material of a microparticle delivery vehicle. In a particular preparation method, the compositions can be isolated and purified by at least one and preferably all steps, involving isolating the microparticles by centrifugation; washing the microparticles with one or more wash cycles; and lyophilizing the microparticles.

In additional embodiments the pharmaceutical compositions can be prepared by any of the above-mentioned methods wherein the stabilizing agent is chosen from at least one of 0-polyethyleneoxides, polysorbates, polyvinylalcohols, polyvinylpyrrolidones, poly(N-2-hydroxypropyl methacrylamide)s, polyhydroxyethylmethacrylates, hydrophilic poly(aminoacid)s such as polylysine or polysaccharides. A particularly preferred polymer additive is polyvinylalcohol (PVA) with 0.5 to 10% w/w PVA blended into the homopolymer or more preferably 1 to 5% w/w PVA. A polymer blend with 2% PVA and 98% homopolymer is particularly preferred.

In preferred embodiments of the present invention, pharmaceutical compositions can be administered subcutaneously for the direct localized treatment of a disease or disorder wherein the pharmaceutical composition includes at least one microparticle with an encapsulated therapeutic agent.

In a particularly preferred embodiment of the present invention, pharmaceutical compositions can be administered intravesically in the lumen of the bladder for the delivery and controlled release of a therapeutic agent for the treatment of a urological disease or disorder. Preferred applications involve intravesicall chemotherapy of a bladder cancer wherein preferred encapsulated therapeutic agents are anticancer drugs.

In specific embodiments, it has been observed that the microparticles of a pharmaceutical composition of the present invention will localize on and adhere to tissues or cellular surfaces where the pharmaceutical composition was administered.

In another embodiment of the present invention, a method for treating a urological disorder wherein the method comprises the step of administering intravesically a microparticle with one or more encapsulated therapeutic agents to the lumen of the bladder wherein the particles localize to the surface of the mucosa where the encapsulated therapeutic agent is delivered to treat the urological disorder with a controlled release from the microparticle. In preferred applications the urological disorder is a cancer and the encapsulated therapeutic agent is an anticancer drug. Particularly preferable applications comprise introducing a pharmaceutical composition of the invention into the lumen of a bladder wherein the microparticles encapsulate paclitaxel.

A specific embodiment is a method for the localized treatment of a disease or disorder comprising of administering a pharmaceutical composition of the invention to the site of a disease or disorder wherein the localized microparticles release the encapsulated therapeutic agent in a controlled release to treat the disease or disorder.

It will be appreciated that the actual preferred amounts of therapeutic agent or other component used in a given composition will vary according to the therapeutic agent being utilized including the polymer system being employed, the mode of application, the particular site of administration, etc. Optimal administration rates for a given protocol of administration can be readily ascertained by those skilled in the art using conventional dosage determination tests conducted with regard to the foregoing guidelines.

As discussed, a preferred mode of application is subcutaneous although for other invention embodiments, more continous administration by stent, catheter or like device may be useful. Another mode of application is topical e.g, when the site of a tumor or metastatic growth has been made accesible by a surgical manipulation.

The present invention is further illustrated by the following examples. These examples are provided to aid in the understanding of the invention and are not to be construed as limitations thereof.

General Comments

Unless otherwise specified, the following materials and methods are used in the examples and elsewhere in this application.

1-ethoxycarbonyl-1-ethoxycarbonylmethylenoxycarbonyl ethene, also referred to as methylidene malonate 2.1.2 (MM 2.1.2) was prepared according to Bru-Magniez et al. (1990). It was kept under sulphur dioxide (SO₂) atmosphere at −18° C. to prevent spontaneous polymerization.

Sodium hydroxide 0.1 M and Paclitaxel were purchased from Sigma. Poly (vinyl alcohol) (88% hydrolyzed) was supplied by Polysciences. Ethyl acetate, acetone and dimethylsulfoxide were used as provided without further purification. Nile Red was supplied by Molecular Probes.

### EXAMPLE 1

### Preparation of Microparticles with Encapsulated Paclitaxel

Paclitaxel-encapsulated PMM 2.1.2 microparticles were prepared by a modified solvent evaporation technique previously described (Bru-Magniez, PCT WO 99/55309). Sulfur dioxide free 1-ethoxycarbonyl-1-ethoxycarbonylmethyleneoxycarbonyl ethene was first dispersed in acetone (1% v/v) and sodium hydroxide (0.1 M) was added to the magnetically stirred acetone dispersion until the sodium hydroxide concentration in acetone was 1%. Polymerization occurred after 5 minutes of stirring and the polymer was recovered after evaporation of the acetone under vacuum. An organic solution of polymer (50 mg in 1.5 mL of ethyl acetate) containing 2.5 mg of paclitaxel (Sigma Chemicals, Inc.) was poured into 15 mL of an aqueous solution of poly(vinylalcohol) (88% hydrolyzed, from Polysciences) (2% w/v) and the emulsification process was conducted during 5 minutes (Polytron PT 1200). The resulting emulsion was then stirred at room temperature during at least 4 hours, until complete evaporation of the ethyl acetate. Hardened microparticles were then isolated by centrifugation, washed 3 times with distilled water then stored at 4° C.

### EXAMPLE 2

### Preparation of Microparticles Without Encapsulated Paclitaxel

Blank microparticles without encapsulated paclitaxel were prepared analogously to Example 1 without adding paclitaxel to the ethyl acetate solution of poly(methylidene malonate).

### EXAMPLE 3

### Preparation of Fluorescent Microparticles

Addition of a hydrophobic fluorescent probe, nile red (1 mg/ml) was dissolved in the ethyl acetate polymer solution in Examples 1 and 2 before the emulsification step. Microparticles were then prepared analogously to Example 1.

### EXAMPLE 4

### Interactions of Paclitaxel with MBT-2 Cells

MBT-2 cells in culture flasks were maintained in Dulbecco's Modified Eagles Medium, 10% fetal calf serum, 100 μg/mL penicillin and 100 μg/mL streptomycin at 37° C. in an atmosphere of 5% CO₂.

For proliferation assay experiments, cells were harvested with Trypsin-EDTA and seeded (3,000 cells) on an uncoated 96-well plate (Falcon). A standard curve was established by using paclitaxel solutions that were prepared by first dissolving paclitaxel in dimethylsulfoxide (DMSO), then dilution with culture medium. Final concentration of DMSO was 0.75%. After 1 day, the culture medium was replaced by medium containing free paclitaxel, with concentrations ranging from 2.9×10⁻⁶ M to 2.9×10⁻⁹ M or paclitaxel extracted from a paclitaxel-loaded microparticle, with serial dilutions. Every day the medium was replaced with fresh medium containing free paclitaxel or extracted paclitaxel. After 3 days, cell proliferation was measured by a tetrazolium-based assay (CellTiter96, Promega). MTS tetrazolium was added to each well (20 μL) and cells were incubated for 4 hours at 37° C. Formazan production, related to enzymatic cell activity, was determined at 490 rim (Biorad plate reader Model 550). Results are expressed as a percentage of growth inhibition compared to cells incubated in the same conditions with medium. Paclitaxel encapsulation was then calculated from the standard curve of growth inhibition of free paxlitaxel.

Paclitaxel-loaded microparticle activity on MBT-2 cells was also determined with no extraction process. Cells were seeded (3,000 cells) on uncoated 96-well plates (Falcon). After 1 day, the culture medium was replaced by a medium containing free paclitaxel or particle suspensions. Every day, medium was replaced with fresh medium containing free paclitaxel or fresh medium only. After 3 days, cell proliferation was measured as described above.

Cells were also observed after incubation with free paclitaxel or particles. Cells were seeded on chambered cover-glasses (Labtek). After 1 day, free paclitaxel (4.4×10⁻⁵ M) or particles (same concentration of paclitaxel) were added. Medium was replaced every day with free paclitaxel or medium only in the case of the particles. On day 4, cells were washed, fixed during 30 minutes with paraformaldehyde (2%) then observed with a confocal microscope (Zweiss Axiovert 100) for localization of particles.

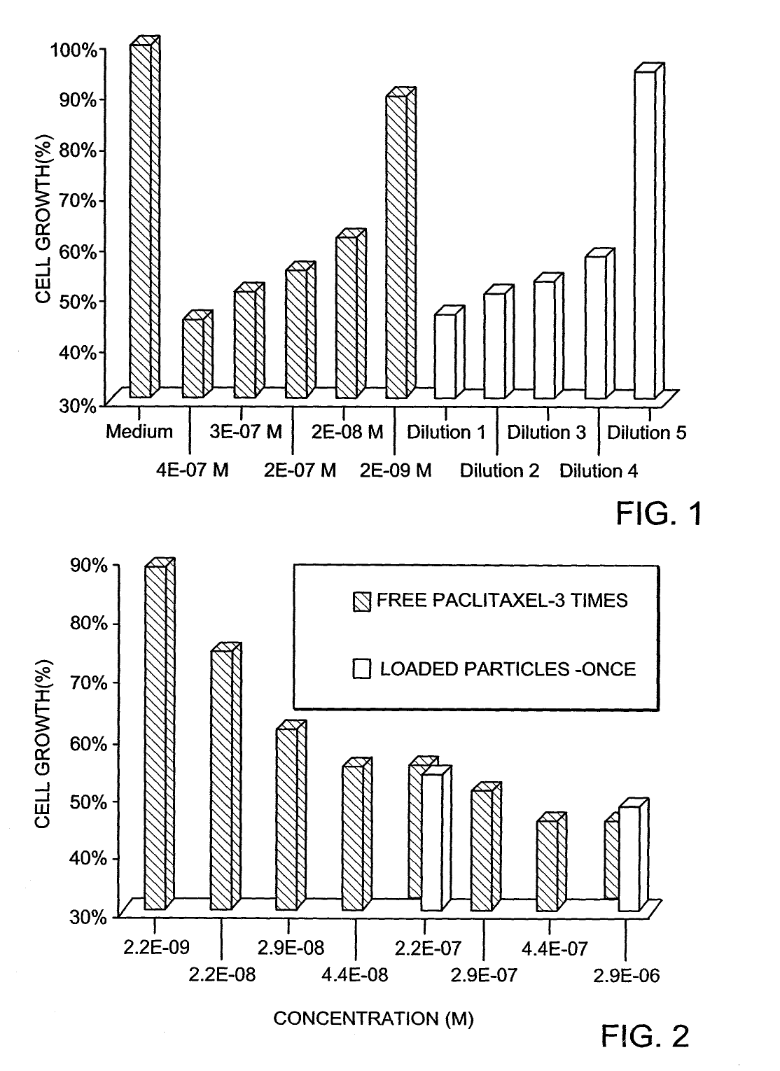

Cell proliferation inhibition with paclitaxel extracted from particles was assessed. MBT-2 cell proliferation decreased with an increased concentration of free paclitaxel in the medium (FIG. 1). With paclitaxel extracted form the microparticles, the same trend was observed, showing that the paclitaxel encapsulated exhibited the same bioactivity in culture. From the standard curve thus obtained, encapsulation level of paclitaxel in microparticles was 5% w/w, corresponding to an encapsulation efficiency of 95%.

Cell proliferation was also dependent on the concentration of paclitaxel encapsulated added to the medium (FIG. 2). With paclitaxel encapsulated in microparticles added only once, the same activity was observed as with free paclitaxel added 3 days. No effect was observed with blank microparticles.

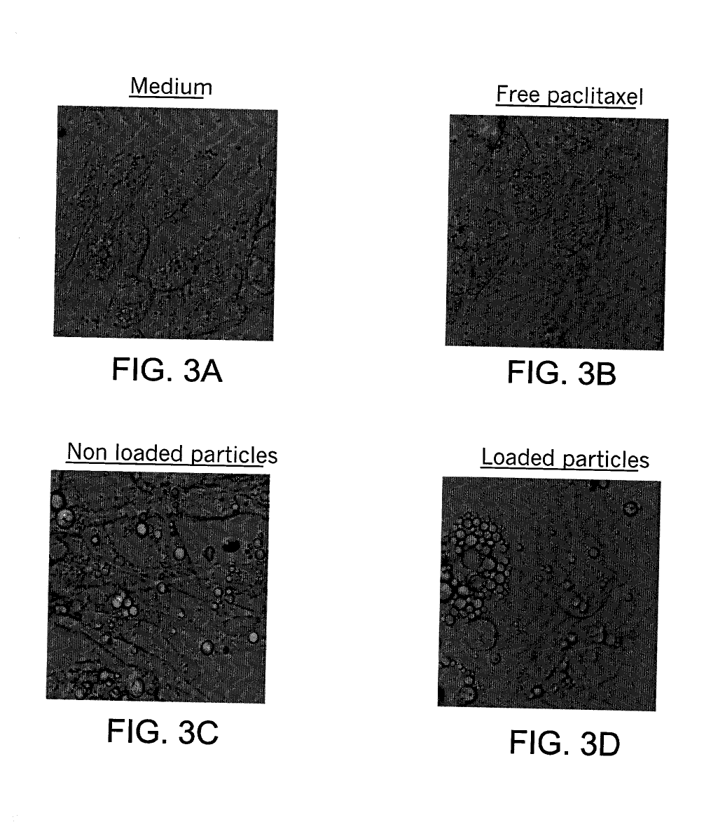

After incubation with free paclitaxel, modification of the shape of the cells was observed, mainly due to the effect of the drug on cytoskeleton (FIG. 3). In the case of loaded particles, the same modification was observed. No effect of blank particles was observed. The microparticles can adhere to the surface of the cells. Using fluorescent particles, the particles were still observed at the surface of the cells after 3 days of incubation and washing cycles.

### EXAMPLE 5

### Distribution of Microparticles After Administration to Mice

Fluorescent microparticles, prepared with nile red, were administered to remail Balb-c mice (8 weeks old) as a single dose intravesically (50 μL). Animals were sacrificed after 30 minutes, 3 and 48 hours. The bladder was excised, fixed in paraformaldehyde 3% during 2 hours then embedded on OCT and frozen. Tissue cryosections (20 μm) (Cryostat) were observed by using a confocal microscope (Zweiss Axiovert 100) with filters for selective FITC excitation (detection of green autofluorescence of the tissues) and selective nile red excitation (for detection of microparticles). Particles were also identified on Hematoxylin and Rosin stained sections. Non-fluorescent particles were also instillated to mice in the same conditions and scanning electron microscopy was preformed on bladder sections to localize particles.

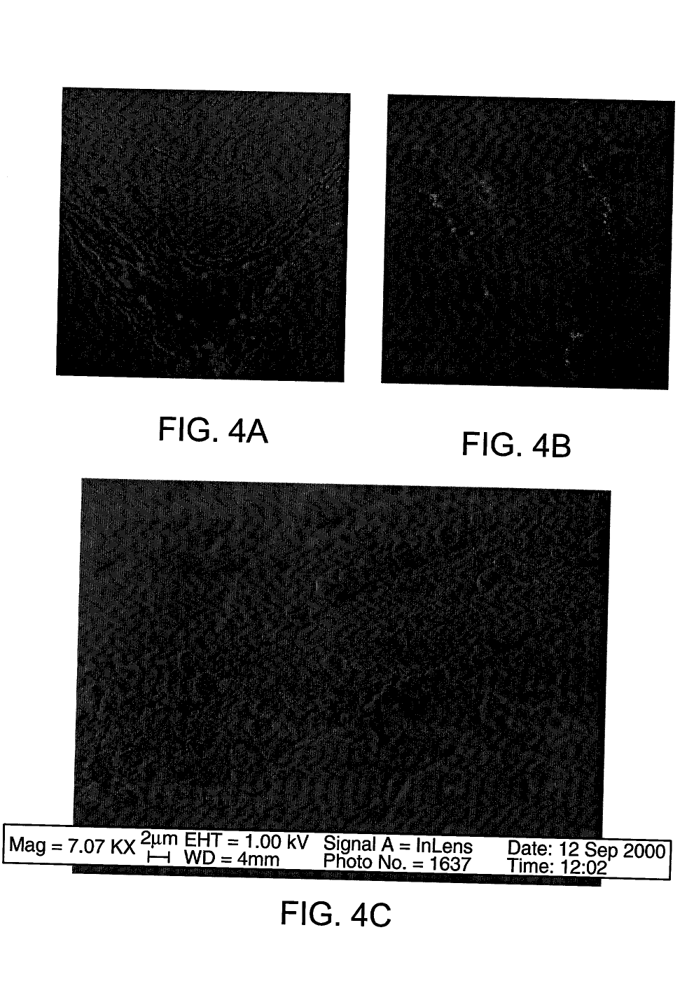

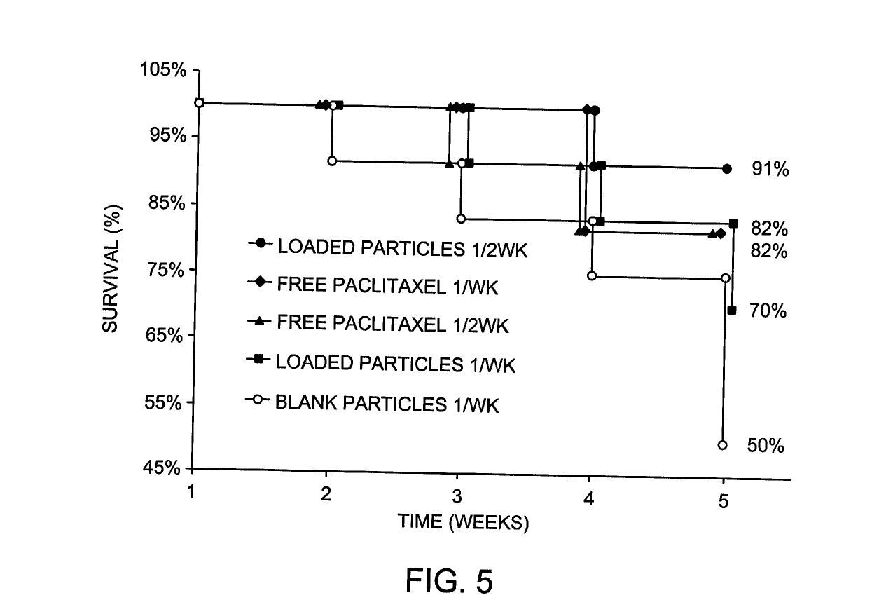

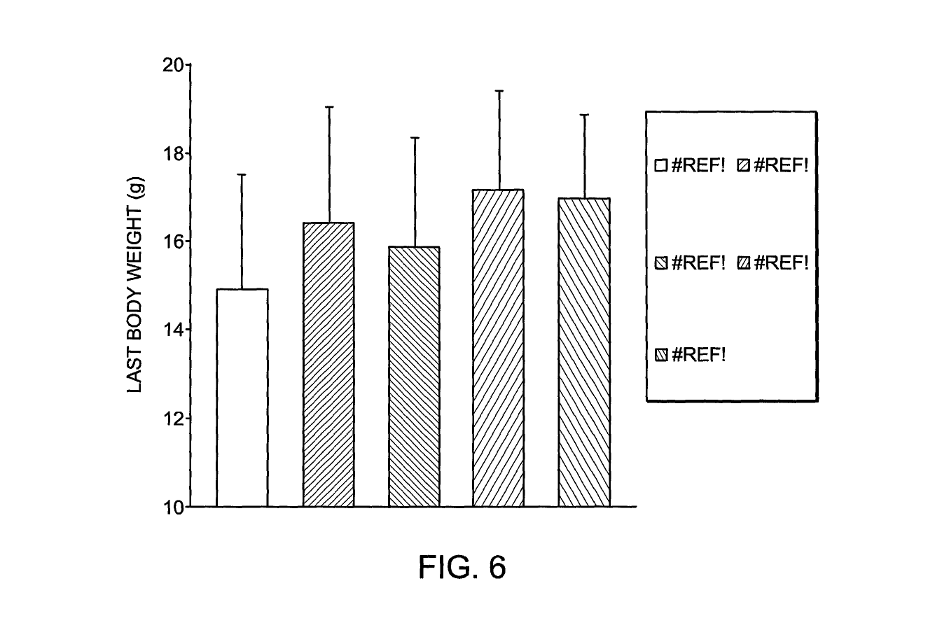

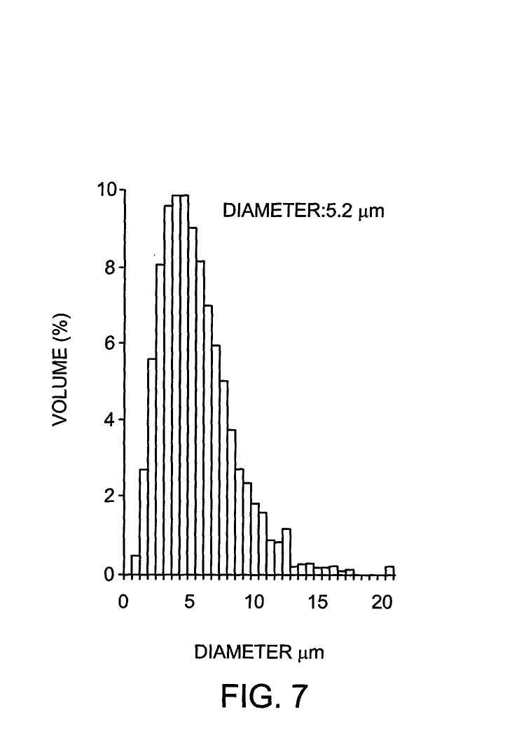

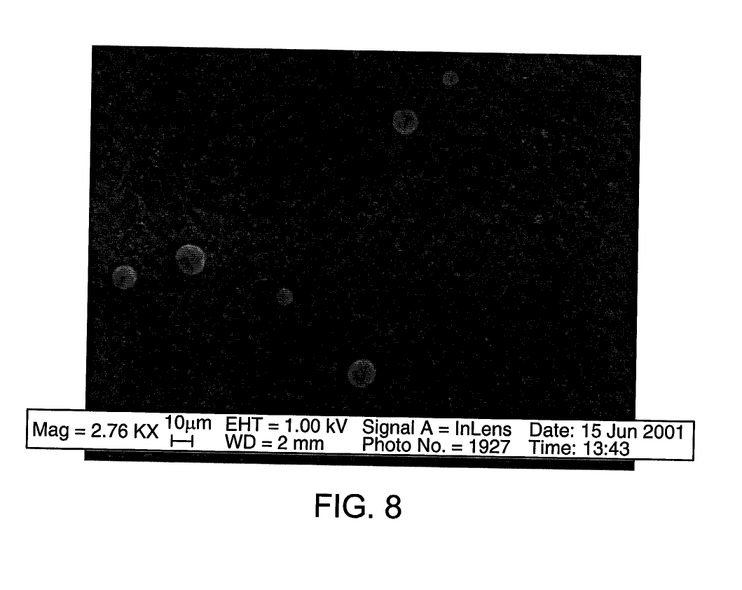

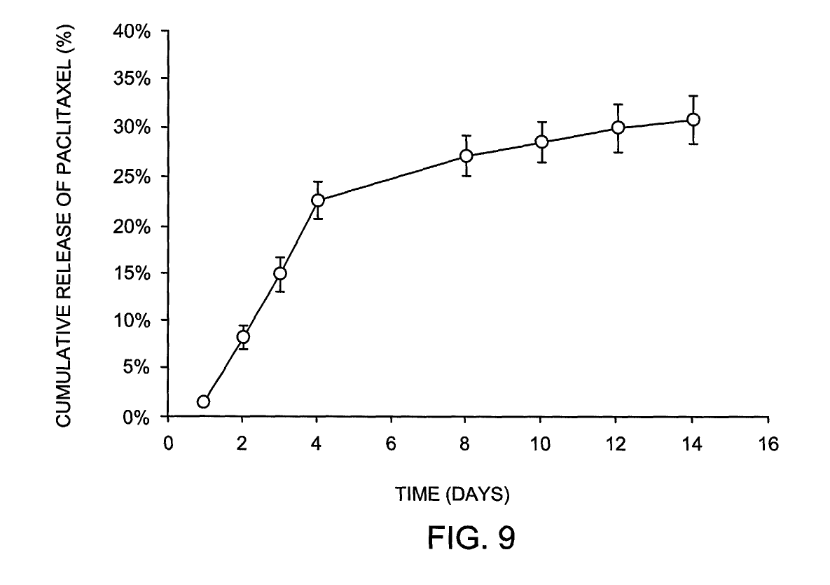

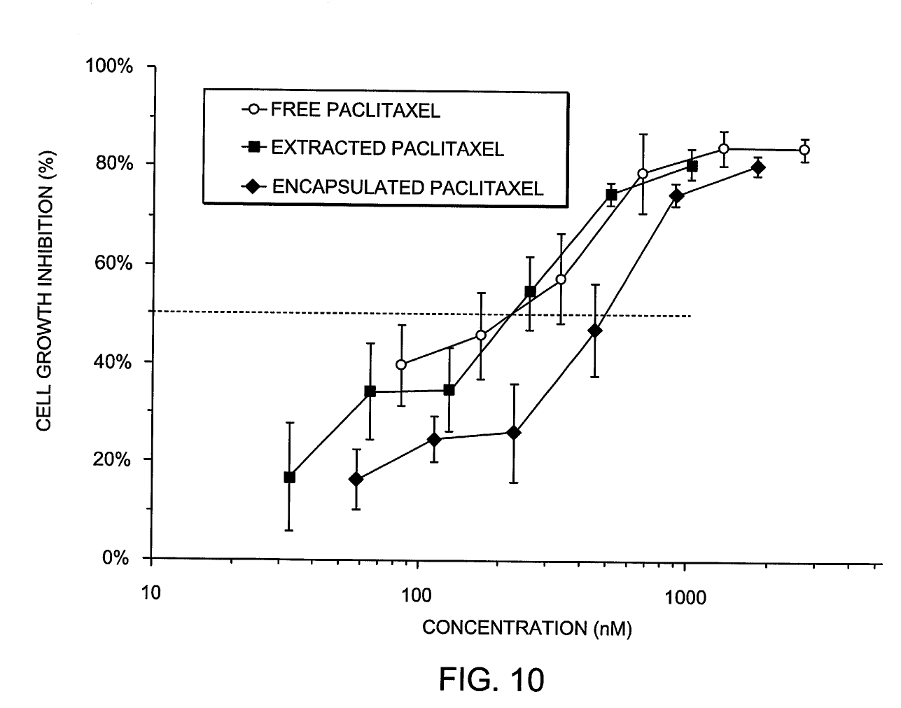

An in vivo observation of microparticles localization in the bladder was performed with scanning electron microscopy. Thirty minutes after the intravesical administration of microparticles encapsulating paclitaxel into mice, particles were observed mainly in the lumen of the bladder (FIG. 4). With scanning electron microscopy, we were able to observe localization of the microparticles at the surface of the mucosa. Surprisingly, particles remained attached to the mucosa after 3 hours and some particles could still be observed attached to the mucosa after 48 hours. These particles can deliver a bioactive molecule specifically to a targeted position such as a bladder mucosa and that a controlled release can be achieved.

### EXAMPLE 6

### Antitumor Activity of Microparticles on Mice Bladder Cancer

Bladder cancer was induced in female Balb-c mice (8 weeks old) with the carcinogen BBN that was given as a 0.05% solution in the drinking water during 4 weeks. Mice were then treated intravesically (50 μL) with free paclitaxel (100 μL) in Tween 80 5%), loaded particles (100 μg) or unloaded particle suspensions. Schedule of administration was the following: mice received the treatment every week during 4 weeks or every other week during 4 weeks. Body weights were recorded every week for all the animals. One week after the last instillation, all the animals were killed. Bladders were removed, weighed and fixed in paraformaldehyde 10% overnight then embedded in paraffin and sectioned.

The antitumor activity of encapsulated paclitaxel was determined. During 4 weeks of treatment with either free or encapsulated paclitaxel, the number of surviving mice as well as the body weight of the mice were recorded. Survival of mice receiving paclitaxel encapsulated (100 μL every other week) was significantly higher than in the other groups (p=0.05). Moreover, the last body weight of these mice was also greater when compared to the controls receiving unloaded microparticles.

Use disclosed as follows of paclitaxel encapsulated in poly (methylidene malonate 2.1.2) microparticles for intravesical administration and local delivery of this anticancer agent.

Paclitaxel, a potent anticancer and antiangiogenic agent, was encapsulated in particles using a single emulsion process. In vitro experiments showed that paclitaxel, after the encapsulation process, retained its biological activity, leading to a decreased growth of culture bladder cells. Moreover, paclitaxel could be released from the particles in vitro with a sustained activity on the cells. After intravesical administration to female Balb-c mice, particles were localized in the lumen of the bladder and remained associated to the mucosa for at least 48 hours.

The antitumor activity of the paclitaxel-loaded microparticles was then assessed using a BBN-induced bladder cancer in mice. After intravesical administration of encapsulated paclitaxel, survival of mice was significantly increased compared to the administration of non-loaded microparticles or the administration of free paclitaxel. Moreover, the last body weight of mice was significantly higher for the mice receiving encapsulated paclitaxel, compared to the non-loaded particles.

Accordingly, provided is a new formulation of paclitaxel, comprising poly (methylidene malonate 2.1.2) microparticles suitably of about 2 micrometers in diameter. After administration to the bladder, the particles remain on the bladder mucosa, leading to a controlled release of the paclitaxel.

### EXAMPLE 7

### Antitumor Activity Evaluations

The anti-cancer activity of paclitaxel encapsulated in microparticles of the invention was assayed against bladder cancer following intravesical instillation. Experimental bladder cancer was induced in female Balb/c mice with BBN, a carcinogenic compound that was added to the drinking water 4 weeks before starting the treatment we compared the same dose (100 μg) of free paclitaxel, encapsulated paclitaxel and non-loaded particles as a control (12 animals per group). Mice received the treatment either once a week or every other week for 4 weeks and number of surviving mice as well as body weight of mice were recorded every week. Significant, differences in survival rates watt obtained between the non-loaded particle control group (50%) and the group that received the paclitaxel-loaded particles every other week (91%) (p<0.05). For the group receiving free paclitaxel once a week or every other week, survival rates were higher. than for the control group (82%) but this was not significantly different Last body weight results showed the same trend, with the control group having the lowest body weight and the groups receiving the loaded particles every other week the highest body weight (p<0.05).

One week after the last instillation, all the animals were sacrificed and histologic evaluation was performed on haematoxilin and eosin stained sections (table 1). Urothelial lesions were observed in all groups, with an hyperplasia resulted from BBN. At least 1 high grade tumor (HGTCC) was identified in each group, in the control group (n=6), all the bladder specimens showed urothelium neoplasia with CIS in 4 cases (67%) and HGTCC in 2 cases (33%). In the group treated with free paclitaxel once a week or every other week, CS was found in 5 cases (71°/b) and 7 cases (78%), respectively. On the contrary, no animal of 7 (0%) in the group treated with encapsulated paclitaxel once a week had CIS. Only 2 animals of 9 (22%) treated with particles every other week had CIS. In the groups treated with the paclitaxel, the urothelium was focally or predominantly denuded. This effect was more pronounced with the particles, with the basal cell layer often visible and exposed to the lumen, but not related to the polymer, as the control group receiving non-loaded particles did not present the same appearance.

Proliferation index was calculated on these sections using the BrdU incroporation into proliferative cells. BrdU staining was localized in the nucleus with a fine granularity. Nuclei were considered BrdU positve if any nuclear staining was observed. The control group presented a proliferative index of 30% compared to the proliferative index of 10% in a normal urothelium. In all other groups, there was a high proliferative index, from about 35% in groups receiving encapsulated paclitaxel to 65% in groups receiving free paclitaxel. Moreover, although the BrdU staining was predominantly observed in the basal cell layer in the group treated with encapsulated paclitaxel, there was a diffuse staining of the urothelial mucosa in the other groups.

Free paclitaxel (100 μg) was shown to have almost no effect on the incidence of CIS in this BBN induced bladder cancer with no difference between the 2 schedules (once a week or every other week). On the contrary, encapsulated paclitaxel (100 μg) was highly effective on decreasing the incidence of CIS. Actually, the more pronounced effect was seen with a weekly administration of the particles, the treatment booing more agressive to the urothelium as seen by the denuded urothelium. Proliferative cells were mainly basal coils in the case of the encapsulated paclitaxel group, indicating a regeneration of the urothelium whereas in other cases the whole urothelium was proliferative.

Previous studies have shown that paclitaxel could be effective on a bladder cancer model using higher dose of paclitaxel, with 6 instillations of 600 μg/mouse. The microparticles dosing was based on the total amount of drug encapsulated, not on the amount of drug actually released to the urothelium Paclitaxel could be relased from the particles and, as the particles remained adsorbed to the bladder mucosa for several days, this allowed a sustained delivery of the drug to the urothelium. This implies that even if the paclitaxel dose available at one time was lower int the case of the particles, the drug remained close to the urothelium and active for several days. This was better than in the case of the free paclitaxel, quickly eliminated in urine. These findings show that the duration of exposure is an important factor in paclitaxel cytotoxicity and that a long-term exposure of tumor cells to low concentrations of paclitaxel may improve the antitumor activity. Results are further indicated below.

TABLE 1
Histologic evaluation of bladder sections of BBN-mice treated
intravesically with blank microparticles, 100 μg of free paclitaxel or 100 μg of
encapsulated paclitaxel either once a week or every other week for 4 weeks.

| | Animals | CIS | HGTCC | Dysplasia | Proliferative |
|---|---|---|---|---|---|
| Instillation | (No) | (No) | (No) | (No) | Index (%) |
| Blank particles (control) | 6 | 4 | 2 | 1 | 30% |
| | | (67%) | | | |
| Free paclitaxel once a week | 7 | 5 | 1 | 0 | 65% |
| | | (71%) | | | |
| Loaded particles once a week | 7 | 0 (0%) | 2 | 0 | 44% |
| Free paclitaxel every other | 9 | 7 | 1 | 1 | 61% |
| week | | (78%) | | | |
| Loaded particles every other | 9 | 2 | 1 | 1 | 35% |
| week | | (22%) | | | |

No: number of animals
CIS: carcinoma in situ
HGTCC: high grade transitional cell carcinoma

### EXAMPLE 8

### Local Delivery of Paclitaxel

An important application of the invention is bladder instillation of PMM 212 microparticles for the local delivery to the urothelium. Such bladder instillation provides activity tumor activity that can not be exaplained by systemic delivery alone of the paclitaxel.

Microparticles encapsulating radiolabeled ³H-Paclitaxel (Moravek Biochemicals, #MT 552, 50 μCi), were prepared following a single emulsion process. Briefly, ³H-Paclitaxel, received as a solution in ethanol, was diluted with a solution of non radiolabeled paclitaxel in ethyle acetate after evaporation of ethanol (300 μg total containing 5 mg of paclitaxel for preparation of 2 batches of microparticles).

Microparticles were then instilled to female Balb/c mice (50 μl of suspension). Reference suspension was kept (50 μl) for the determination of the total amount of radioactivity instilled. Animals wee sacrificed after 4, 5, 6 and 7 days (2 animals per time point). Blood and urine samples were collected, and liver, kidneys, spleen, lungs, heart were removed. Samples were weighted, then, up to 20 mg of minced tissue were placed in a scintillation vial and dissolved using a tissue solubilizer (Biosol) for 2 days. After digestion, the samples were discolored with 0.2 ml of a 30% H202 solution and mixed with 10 ml of a scintillating cocktail (Bioscint). Samples were counted on a Liquid scintillation counter (Beckman). Results are expressed in Tables 2 and 3 below as a percentage of the instilled that was found in each sample as well as the amount of paclitaxel.

TABLE 2
Pharmacokinetics of encapsulated radiolabeled paclitaxel after bladder
instillation to female Bal/c mice: percentage of the instilled dose.

| Sample | Day 4 | Day 5 | Day 6 | Day 7 |
|---|---|---|---|---|
| Blood | 0.4% | 0.3% | 0.0% | 0.0% |
| Urine | 0.2% | 0.0% | 0.0% | 0.1% |
| Liver | 1.8% | 0.5% | 0.2% | 0.0% |
| Spleen | 0.3% | 0.0% | 0.0% | 0.7% |
| Kidneys | 0.6% | 0.2% | 0.5% | 0.2% |
| Heart | 0.1% | 0.1% | 0.0% | 0.0% |
| Lungs | 0.5% | 0.1% | 0.3% | 0.0% |

TABLE 3
Pharmacokinetics of encapsulated radiolabeled paclitaxel after bladder
instillation to female Bal/c mice; amount of paclitaxel (μg).

| Sample | Day 4 | Day 5 | Day 6 | Day 7 |
|---|---|---|---|---|
| Blood | 0.4% | 0.3% | 0.0% | 0.0% |
| Urine | 0.2% | 0.0% | 0.0% | 0.1% |
| Liver | 1.8% | 0.5% | 0.2% | 0.0% |
| Spleen | 0.3% | 0.0% | 0.0% | 0.7% |
| Kidneys | 0.6% | 0.2% | 0.5% | 0.2% |
| Heart | 0.1% | 0.1% | 0.0% | 0.0% |
| Lungs | 0.5% | 0.1% | 0.3% | 0.0% |

### EXAMPLE 9

### Local Gene Delivery

Microparticles encapsulating various plasmids were prepared according to the following process. The first emulsion was prepared using 150 μl of a solution of plasmid in water (about 10 mg/ml) that was emulsified in the organic phase containing 50 mg of polymer dissolved in ethyl acetate and sonicated for 15 seconds. The second emulsion was formed by adding 15 mil of PVA 2% and homogenized (Polytron PT1200 homogenizer) on speed 6 for 5 minutes. The mixture was allowed to stir overnight to evaporate ethyl acetate and followed by 5 washes of deionized water and centrifugation for 5 minutes to collect the particles that wee stored in water at 4° C. until use. DNA concentration in supernatants from the washing steps was measured using fluorimetry after complexion of DNA with Hoechst 33258. Encapsulation rate was then calculated to be of 0.5%.

For direct fluorescent visualization of transfection, DS-Red DNA encoding for Red Fluorescent Protein (RFP) was used. Particle suspension, containing approximately 25 leg of DNA, was instilled into female Balb/e mice. Following sacrifice of the mice, bladders were removed, frozen on dry ice and sliced into 10 micron sections on a cryomicrotome. Tissue sections were mounted to a slide with Gelmount containing anti-fading reagents and observed with a confocal microscope.

DNA transfection was observed 2 days after instillation, with isolated rod cells observed on different bladder sections. Only urothelial cells were observed to be transfected, with no red cells observed in the bladder wall.

For quantification of the expression, SEAP plasmid, encoding for the human secreted alkaline phosphatase was encapsulated into PMM 2.1.2 microparticles. Particle suspension (50 μl) was instilled into female Balb/c mice. After 1 and 2 days, SEAP activity in urine samples was measured using a substrate for this enzyme and SEAP concentration in the samples was calculated from a standard curve using a SEAP solution. Results are set forth in Tavles 4 and 5 below.

TABLE 4
Secreted Alkaline Phosphatase concentration (ng/ml) in urine 1 day
after bladder instillation of 5EAP plasmid (25 μg) either as a solution
in water or encapsulated in PMM 2.1.2 microparticles.

| | | PMM 2.1.2 |
|---|---|---|
| Plasmid (μg) | Plasmid solution | microparticles |
| 25 | 8.7 | 6.7 |

TABLE 5
Secreted Alkaline Phosphatase concentration (ng/ml) in urine 2
days after bladder instillation of SEAP plasmid (1, 10, 25 or 50 μg) either
as a solution in water or encapsulated in PMM 2.1.2 microparticles.

| | | PMM 2.1.2 |
|---|---|---|
| Plasmid (μg) | Plasmid solution | microparticles |
| 1 | 7.8 | 1.2 |
| 10 | 4.3 | 4.7 |
| 25 | 7.3 | 4.1 |
| 50 | 5.5 | 5.0 |

Although a preferred embodiment of the invention has been described using specific terms, such description is for illustrative purposes only, and it is to be understood that changes and variations may be made without departing from the spirit or scope of the following claims.

Bru-Magniez, N., De Cock, C., Poupaert, J., De Keyser, J. L., and Dumont, P. Process for the preparation of monoesters or diesters of 9,10-endoethano-9,10-dihydroanthracene-11,11-dicarboxylic acid, novel monoesters or diesters prepared by this process and use thereof for the preparation of symmetrical or asymmetrical methylidenemalonates U.S. Pat. No. 4,931,584, 1990.

Bru-Magniez, N., Le Visage, C., Fattal, E., Couvreur, P., and Breton, P. Novel poly (methylidene malonate) microspheres, preparation method and pharmaceutical compositions containing them Int'l Patent PCT, WO, 99/55309, 1999.

Song D., Wientjes, M. G., Au, J. Bladder tissue pharmacokinetics of intravesical taxol, Cancer Chemother. Pharmacol. (1997) 40; 285-292

Duque, J., Loughlin, K. Superficial bladder cancer: new strategies in diagnosis and treatment, Urologic clinics of North America (2000) 27; 125-135

All references disclosed herein are incorporated by reference.

What is claimed is:

1. A pharmaceutical composition comprising:
a microparticle that includes a polymeric support material in which a substance can be dispersed, wherein the support material comprises at least about 50% w/w of at least one homopolymer with a repeat unit according to Formula (I): 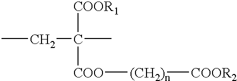
wherein
R₁ represents a C₁-C₆ alkyl group or a group (CH₂)ₘ—COOR₃ wherein m is an integer from 1 to 5 and R₃ is a C₁-C₆ alkyl group, R₁ and R₃ being the same or different;
R₂ represents a C₁-C₆ alkyl group the same or different from R₁ and R₃;
n is an integer from 1 to 5; and
at least one therapeutic agent that is encapsulated or dispersed in the polymeric support material of the microparticle.

2. A pharmaceutical composition according to claim 1 wherein:
R₁ and R₂ are independently chosen C₁-C₆ alkyl groups; and
n is 1.

3. A pharmaceutical composition according to claim 1 wherein:
the stated homopolymer comprising repeat units according to Formula (I) wherein R₁ and R₂ are ethyl groups; and
n=1.

4. A pharmaceutical composition according to claim 3, wherein the composition being obtained by a single emulsification process.

5. A pharmaceutical composition according to any one of claims 1 to 4 wherein the support material comprises:
from about 90 to about 99.5% by weight of a homopolymer as defined in claims 1, 2, or 3; and
from about 0.5 to about 10% by weight of a polymer additive.

6. A pharmaceutical composition according to claim 5 wherein the polymer additive comprises at least one of polyethyleneoxide, polyvinylalcohol, polyvinylpyrrolidone, poly(N-2-hydroxypropyl methacrylamide), polyhydroxyethylmethacrylate, hydrophilic poly(aminoacid) such as polylysine or polysaccharide.

7. A pharmaceutical composition according to claims 5 and 6 wherein the polymer additive is a polyvinylalcohol.

8. A pharmaceutical composition according to any one of claims 1 through 7 wherein the dispersed substance is hydrophobic.

9. A pharmaceutical composition according to any one of claims 1 through 8 wherein the dispersed substance is a therapeutic agent that requires a solvation vehicle for administration.

10. A pharmaceutical composition according to any one of claims 1 through 7 wherein the dispersed substance is hydrophylic.

11. A pharmaceutical composition according to any one of claims 1 to 10, wherein the dispersed substance is a therapeutic agent.

12. A pharmaceutical composition according to any one of claims 1 through 10 wherein the dispersed substance is a peptide or polypeptide.

13. A pharmaceutical composition according to claims 1 through 12 wherein the dispersed substance is a protein.

14. A pharmaceutical composition according to any one of claims 1 through 13 wherein the dispersed substance is a bioactive molecule such as a drug, a therapeutic agent, an anticancer agent, a gene therapy agent, a plasmid DNA, a protein, an enzyme, a peptide, a radionuclide, a protein inhibitor, an analgesic, an anti-inflamatory agent, an antibiotic, an antiviral agent, an antineoplastic agent, a pyrimidine, purine or folic acid analog, an cytotoxic agent, an immunomodulator, a hormone, an antibody or a painkiller.

15. The pharmaceutical composition of claim 14 wherein the pyrimidine analog is fluorouracil (5-FU).

16. A pharmaceutical composition according to any one of claims 1 through 15 wherein the dispersed substance is a bioactive molecule such as an anticancer agent or a gene therapy agent.

17. A pharmaceutical composition according to any one of claims 1 through 16 wherein the dispersed substance is a therapeutic agent for treating or reducing the severity of a urological disease or disorder.

18. A pharmaceutical composition according to any one of claims 1 through 17 wherein the dispersed substance is a therapeutic agent for bladder cancer.

19. A pharmaceutical composition according to any one of claims 1 through 18, wherein the dispersed substance is a taxane.

20. A pharmaceutical composition according to claim 19, wherein the taxane is paclitaxel, docetaxel (Taxotere®) or taxol®.

21. A method of preparing a pharmaceutical composition according to any one of claims 1 through 20 wherein the dispersed therapeutic is hydrophobic comprising the steps of:
a) preparing a first solution in a volatile organic solvent wherein the solution comprises a polymeric support material and a therapeutic agent;
b) preparing a second solution immiscible with the first solution, the second solution comprising a stabilizing agent;
c) preparing an emulsion by combining the first and second solutions sufficient to produce a single phase being composed of a polymer solution; and
d) evaporating the volatile organic solvent while stirring the emulsion to make the pharmaceutical composition.

22. A method of preparing a pharmaceutical composition according to any one of claims 1 through 20 wherein the dispersed therapeutic is hydrophilic comprising the steps of:
a) preparing a first solution in a volatile organic solvent wherein the solution comprises a polymeric support material;
b) preparing a second aqueous solution immiscible with the first solution, the second solution comprising a stabilizing agent and the therapeutic agent;
c) preparing an emulsion by combining the first and second sufficient to produce a single phase being composed of a polymer solution; and
d) evaporating the volatile organic solvent while stirring the emulsion to make the pharmaceutical composition.

23. A method according to claim 21 or claim 22, wherein the method comprises the addition steps:
e) isolating the pharmaceutical composition by centrifugation; and
f) washing the pharmaceutical composition with one or more wash cycles;

24. A method according to any one of claims 21 through 23 wherein the method comprises the addition step of:
(h) lyophylizing the microparticles.

25. A method according to any of claims 21 through 24, wherein the polymer support material is a poly(methylidene malonate 2.1.2).

26. A method according to any of claims 21 through 25, wherein the stabilizing agent is chosen from polyethyleneoxides, polysorbates, polyvinylalcohols, and polymer additives described in claims 5 and 6.

27. A method according to any one of claims 21 through 26 wherein the stabilizing agent is a polyvinylalcohol.

28. Use of a pharmaceutical composition for the preparation of a medicament intended for the localized treatment of a disease or disorder wherein the pharmaceutical composition includes at least one microparticle according to claims 1 through 20 or prepared by a method according to claims 21 through 27.

29. Use of a pharmaceutical composition of any one of claims 1 through 20 for treatment of a urological disease or disorder.

30. The use of claim 29 wherein the pharmaceutical composition provides for controlled release of a therapeutic agent.

31. The use of claim 29 wherein the therapeutic agent to be delivered in a controlled release is an anticancer drug for the treatment of bladder cancer.

32. The use of claims 28 through 31 wherein the microparticles adhere to cells of the tissue where the pharmaceutical composition was administered.

33. Use of a pharmaceutical composition of any one of claims 1 through 20 for treatment of cancer.

34. Use of a pharmaceutical composition of any one of claims 1 through 20 for treatment of bladder cancer.

35. A method of treating a subject suffering from or susceptible to a urological disease or disorder, comprising administering to the subject an effective amount of a pharmaceutical composition of any one of claims 1 through 20.

36. A method of treating a subject suffering from or susceptible to cancer, comprising admistering to the subject an effective amount of a pharmaceutical composition of any one of claims 1 through 20.

37. A method for treating a urological disorder comprising:
administering intravesically a microparticle with one or more encapsulated therapeutic agents to the lumen of the bladder, contacting the particles to the surface of the mucosa, releasing the encapsulated therapeutic agent in a controlled manner to treat the urological disorder.

38. A method according to claim 37 wherein the microparticle comprises a poly(methylidene malonate 2.1.2) polymer support material.

39. A method according to claim 37 wherein the urological disorder is a cancer and the microparticle encapsulated therapeutic agent is an anticancer agent.

40. A method according to any one of claims 37 through 39 wherein the anticancer agent is a taxane.

41. A method according to any to claim 40 wherein the taxane is paclitaxel, docetaxel (Taxotere®) or taxol®.

42. A method according to any one of claims 37 through 41, wherein microparticles with encapsulated paclitaxel are used for intravesical chemotherapy of bladder cancer.

43. A method for the localized treatment of a disease or disorder comprising the steps of: administering a pharmaceutical composition according to claims 1 through 20 to the site of a disease or disorder, contacting the microparticles with the site, and releasing the encapsulated therapeutic agent in a controlled manner to treat the disease or disorder.
